(19) United States
(12) Patent Application Publication
(10) Pub. No.: US 2004/0082516 A1
(43) Pub. Date: Apr. 29, 2004

(54) Compositions for promoting nerve regeneration

(75) Inventors: Marc Tessier-Lavigne, San Francisco, CA (US); Kuan Hong Wang, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA

(21) Appl. No.: 10/721,407

(22) Filed: Nov. 24, 2003

Related U.S. Application Data
(63) Continuation of application No. 09/273,098, filed on Mar. 19, 1999, now abandoned.
(60) Provisional application No. 60/125,591, filed on Mar. 18, 1999.

(51) Int. Cl. A61K 31/17; C07K 14/705

(52) U.S. Cl. 514/12; 530/350

(57) ABSTRACT

Slit-N polypeptides are used to promote axon branching and nerve regeneration in vitro and in vivo. Pharmaceutical compositions comprising a therapeutically effective amount of a Slit-N polypeptide, a pharmaceutically acceptable excipients and/or neuroactive agents, particularly neurotrophins such as NGF are used to treat neuropathies.

### CROSS-REFERENCE TO RELATED APPLICATIONS

This application claims priority to U.S. application, Ser. No. (not yet assigned), having the same title and inventors, filed Mar. 18, 1999. Slit compositions are subject to claims in copending U.S. application, Ser. No. 09/191,647, having the same assignee:

### INTRODUCTION TO INVENTION

1. Field of the Invention

The field of this invention is compositions for promoting nerve regeneration

2. Background

Many neurons in both vertebrates and invertebrates innervate multiple targets by sprouting secondary axon collaterals (or branches) from a primary axon shaft. To identify molecular regulators of axon branch initiation or extension (Tessier-Lavigne and Goodman ((1996) Science 274, 1123-33.), we studied the growth of single sensory axons in an in vitro collagen assay system, and identified an activity in extracts of embryonic spinal cord and of postnatal and adult brain that promotes the elongation and formation of extensive branches by these axons. Using biochemical purification of the activity from calf brain extracts, we identified of an amino-terminal fragment of Slit-2 as the main active component (see, Wang et al., 1999, Cell 96, 771-784). We disclose that N-terminal fragments of Slit proteins (see Kidd et al. (1999) Cell 96, 785-794; Brose et al. (1999) Cell, 795-806; and Li et al. (1999) Cell, 807-818), can function as positive regulators of axon collateral formation during the establishment or remodeling of neural circuits and that the activity of these proteins can synergize in vitro and in vivo with other neurotrophic agents like NGF. We find that Slit-N proteins can function to regulate axon collateralization not just during the initial development of axonal connections, but also during normal plastic rearrangements of neural connections that occur in the adult nervous system. Following injury to the spinal cord, Slit-N proteins can induce regeneration by stimulating collateralization of axons from fiber tracts into the CNS gray matter, and/or axon regrowth in an inhibitory environment, to help alleviate the paralysis that accompanies injury to fiber tracts.

### SUMMARY OF INVENTION

The invention provides methods and compositions for promoting axon branching and nerve regeneration. The compositions comprise Slit-N polypeptides including Slit-1-N, Slit-2-N and Slit-3-N polypeptides. Slit-N polypeptides are proteolytic fragments of Slit proteins, which stimulate elongation and branching of neuronal axons. Accordingly, the invention also provides-pharmaceutical compositions comprising a therapeutically effective amount of one or more Slit-N polypeptide. Such compositions may also comprise pharmaceutically acceptable excipients and/or neuroactive agents, particularly neurotrophins such as NGF. The Slit-N polypeptides are generally prepared from cells expressing a recombinant polynucleotide comprising a coding region encoding a Slit-N polypeptide. The subject methods include methods of promoting axon branching or sprouting, comprising contacting a neuron with a composition comprising an effective amount of a Slit-N polypeptide and methods of treating a neuropathy comprising administering a composition comprising a therapeutically effective amount of a Slit-N polypeptide.

### DESCRIPTION OF PARTICULAR EMBODIMENTS OF INVENTION

The following descriptions of particular embodiments and examples are offered by way of illustration and not by way of limitation. Unless contraindicated or noted otherwise, in these descriptions and throughout this specification, the terms “a” and “an” mean one or more, the term “or” means and/or and polynucleotide sequences are understood to encompass opposite strands as well as alternative backbones described herein.

The compositions find used (1) assisting repair of the nervous system following injury or trauma, such as spinal cord injury, and (2) alleviating dysfunction of the nervous system due to hypertrophy of neurons or their axonal projections, or other dysfunctions of neuronal populations, such as occurs in diabetic neuropathy.

Slit-N polypeptides encompass N-terminal fragments of Slit proteins and which promote axon branching in the cell and in vivo methods described herein. Slit proteins are an art-recognized class of neuroactive proteins. Preferred Slit-N proteins used in the disclosed methods derive from mammalian Slit sequences, preferably human Slit-1, human Slit-2 or human Slit-3 sequences. In one embodiment, the Slit-N polypeptides are derived from cells expressing recombinant Slit proteins, which then proteolytically process the Slit proteins to form the Slit-N polypeptides. Any empirically functional expression/processing systems may be used, preferably mammalian cell-based systems, such as mammalian CHO or COS cells.

Alternatively, the Slit-N polypeptides can be obtained from cells expressing recombinant Slit-N polypeptides directly. Any empirically functional expression system may be used, including well-established commercial animal cell (e.g. CHO cells, COS cells, Baculovirus-based systems, etc.) and microbial systems, such a S. cerevisiae and E. coli systems. The expression construct will encode a Slit-N polypeptide having of a natural Slit-N sequence. Such sequences are readily ascertained by sequencing naturally processed Slit proteins: for example, when recombinant human Slit-2 is expressed in mammalian (COS) cells, in addition to a full length protein migrating at ˜190 kD, an amino terminal cleavage product (Slit-2-N) is observed migrating at ˜140 kD, similar to p140, and a carboxy terminal cleavage product (Slit-2-C) is observed migrating at ˜55-60 kD. Most of the full length protein and Slit-2-N are associated with cell membranes, but can be extracted with 1 M NaCl. The C-terminal fragment is mainly secreted into conditioned media. Termini amino acid sequencing of the Slit-2-N can be performed directly by standard methods. Alternatively, Slit-2-N sequences may be inferred from amino acid sequencing of the amino terminus of Slit-2-C, which reveals a natural processing site between Arg1117 and Thr1118. In addition, natural Slit-N sequences can be predicted by sequence alignment with homologs, such as hSlit-2-N, wherein a natural hSlit-2-N sequence is bound by Met1 and Arg1117; for example, alignment of hSlit-2-N with dSlit-2 (drosophila) reveals the corresponding predicted dSlit-2-N to be dSlit-2, Met1-Gln1111. Natural Slit-N sequences derived from such inferences or predictions are confirmed by direct amino acid analysis.

The encoded Slit-N polypeptide may also comprise a deletion mutant of a natural Slit-N sequence so long as the requisite axon branching promoting activity is retained. The encoded Slit-N polypeptides may also be addition mutants, which comprise in addition to a natural Slit-N sequence, additional Slit or non-slit residues, substitution mutants, which comprise one to 20, preferably one to 5, residue substitutions, preferably conservative substitutions from the natural Slit-N sequence, or a combination deletion, addition and/or substitution mutant. Such deletion, addition and substitution mutants are readily screened in the methods described herein. For example, a number of active Slit-N deletion, addition and substitution mutants are shown in Tables 1 and 2.

TABLE 1
Active Slit-N deletion mutants

| | axon branching | | axon branching |
|---|---|---|---|
| Slit-N polypeptide | activity | Slit-N polypeptide | activity |
| hSlit-2-N(Met1-Pro1116) | +++ | dSlit-2-N(Met1-Pro1110) | +++ |
| hSlit-2-N(Met1-Leu1115) | +++ | dSlit-2-N(Met1-Tyr1109) | +++ |
| hSlit-2-N(Met1-Val1114) | +++ | dSlit-2-N(Met1-Met1108) | +++ |
| hSlit-2-N(Arg2-Arg1117) | +++ | dSlit-2-N(Ala2-Gln1111) | +++ |
| hSlit-2-N(Arg2-Pro1116) | +++ | dSlit-2-N(Ala2-Pro1110) | +++ |
| hSlit-2-N(Arg2-Leu1115) | +++ | dSlit-2-N(Ala2-Tyr1109) | +++ |
| hSlit-2-N(Arg2-Val1114) | +++ | dSlit-2-N(Ala2-Met1108) | +++ |
| hSlit-2-N(Gly3-Arg1117) | +++ | dSlit-2-N(Ala3-Gln1111) | +++ |
| hSlit-2-N(Gly3-Pro1116) | +++ | dSlit-2-N(Ala3-Pro1110) | +++ |
| hSlit-2-N(Gly3-Leu1115) | +++ | dSlit-2-N(Ala3-Tyr1109) | +++ |

TABLE 2
Active Slit-N addition and substitution mutants

| Slit-N polypeptide | axon branching | Slit-N polypeptide | axon branching |
|---|---|---|---|
| (internal substitutions) | activity | (N/C termini additions) | activity |
| hSlit-2-N(Leu → Ile1115) | +++ | hSlit-2-N(+N-AspArgGly) | +++ |
| hSlit-2-N(Val → Ile1114) | +++ | hSlit-2-N(+C-His) | +++ |
| hSlit-2-N(Ser → Thr1110 | +++ | hSlit-2-N(+N-AspArgGly) | +++ |
| hSlit-2-N(Phe → Try1109) | +++ | hSlit-1-N(+C-His) | +++ |
| hSlit-2-N(Glu → Asp1108) | +++ | hSlit-1-N(+N-AspArgGly) | +++ |
| dSlit-2-N(Gln → Asn1111) | +++ | hSlit-1-N(+C-His) | +++ |
| dSlit-2-N(Tyr → Phe1109) | +++ | mSlit-2-N(+N-AspArgGly) | +++ |
| dSlit-2-N(Met → Ser1108) | +++ | mSlit-2-N(+C-His) | +++ |
| dSlit-2-N(Met → Ser1107) | +++ | mSlit-1-N(+C-AspArgGly) | +++ |
| dSlit-2-N(Ser → Thr1106) | +++ | mSlit-1-N(+C-HisHis) | +++ |

The subject polypeptides can also be expressed in cell and cell-free systems (e.g. Jermutus L, et al., Curr Opin Biotechnol. 1998 October; 9(5):534-48) from encoding polynucleotides, such as naturally-encoding Slit-N encoding genes and gene transcripts known in the art and/or isolated with degenerate oligonucleotide primers and probes generated from Slit-N polypeptide sequences (“GCG” software, Genetics Computer Group, Inc, Madison Wis.) or polynucleotides optimized for selected expression systems made by back-translating the subject polypeptides according to computer algorithms (e.g. Holler et al. (1993) Gene 136, 323-328; Martin et al. (1995) Gene 154, 150-166).

Accordingly, the invention also provides natural and synthetic sequence polynucleotides encoding Slit-N polypeptides. Such polynucleotides having a naturally occurring Slit-N coding region are isolated, i.e. unaccompanied by at least some of the material with which it is associated in its natural state, preferably constituting at least about 0.5%, preferably at least about 5% by weight of total nucleic acid present in a given fraction, and usually recombinant, meaning they comprise a non-natural sequence or a natural sequence joined to nucleotide(s) other than that which it is joined to on a natural chromosome. Recombinant polynucleotides contain such natural sequence at a terminus, immediately flanked by (i.e. contiguous with) a sequence other than that which it is joined to on a natural chromosome, or flanked by a native flanking region fewer than 10 kb, preferably fewer than 2 kb, more preferably fewer than 500 bases, most preferably fewer than 100 bases, which is at a terminus or is immediately flanked by a sequence other than that which it is joined to on a natural chromosome. While the nucleic acids are usually RNA or DNA, it is often advantageous to use nucleic acids comprising other bases or nucleotide analogs to provide modified stability, etc.

For in situ applications, compositions comprising Slit-N polypeptides may be administered by any effective route compatible with therapeutic activity of the compositions and patient tolerance. For CNS administration, a variety of techniques are available for promoting transfer of the therapeutic across the blood brain barrier including disruption by surgery or injection, drugs which transiently open adhesion contact between CNS vasculature endothelial cells, and compounds which facilitate translocation through such cells. Exemplary routes of administration include direct injection or infusion, topical, intratracheal/nasal administration e.g. through aerosol, intraocularly, or within/on implants e.g. fibers e.g. collagen, osmotic pumps, grafts comprising appropriately transformed cells, etc. A particular method of administration involves coating, embedding or derivatizing fibers, such as collagen fibers, protein polymers, etc. with the subject therapeutic compositions. Other useful approaches are described in Otto et al. (1989) J Neuroscience Research 22, 83-91 and Otto and Unsicker (1990) J Neuroscience 10, 1912-1921. Generally, the amount administered will be empirically determined, typically in the range of about 10 to 1000 μg/kg of the recipient and the concentration will generally be in the range of about 50 to 500 μg/ml in the dose administered. Other additives may be included, such as stabilizers, bactericides, etc. will be present in conventional amounts.

In one embodiment, the invention provides administering the subject Slit-N polypeptides in combination with a pharmaceutically acceptable excipient such as sterile saline or other medium, gelatin, an oil, etc. to form pharmaceutically acceptable compositions. The compositions and/or compounds may be administered alone or in combination with any convenient carrier, diluent, etc. and such administration may be provided in single or multiple dosages. Useful carriers include solid, semi-solid or liquid media including water and non-toxic organic solvents. As such the compositions, in pharmaceutically acceptable dosage units or in bulk, may be incorporated into a wide variety of containers, which may be appropriately labeled with a disclosed use application. Dosage units may be included in a variety of containers including capsules, pills, etc. The compositions may be advantageously combined and/or used in combination with other therapeutic or prophylactic agents, different from the subject compounds. In many instances, administration in conjunction with the subject compositions enhances the efficacy of such agents, see e.g. Goodman & Gilman's The Pharmacological Basis of Therapeutics, 9th Ed., 1996, McGraw-Hill. Exemplary such other therapeutic agents include neuroactive agents such as in Table 3.

TABLE 3
Neuroactive agents which may be used in
conjunction with Slit-N polypeptides.

| | | |
|---|---|---|
| NGF | Heregulin | Laminin |
| NT3 | IL-3 | Vitronectin |
| BDNF | IL-6 | Thrombospondin |
| NT4/5 | IL-7 | Merosin |
| CNTF | Neuregulin | Tenascin |
| GDNF | EGF | Fibronectin |
| HGF | TGFa | F-spondin |
| bFGF | TGFb1 | Netrin-1 |
| LIF | TGFb2 | Netrin-2 |
| IGF-I | PDGF BB | Semaphorin-III |
| IGH-II | PDGF AA | L1-Fc |
| Neurturin | BMP2 | NCAM-Fc |
| Percephin | BMP7/OP1 | KAL-1 |

Abbreviations: NGF, nerve growth factor; NT, neurotrophin; BDNF, brain-derived neurotrophic factor; CNTF, ciliary neurotrophic factor; GDNF, glial-derived neurotrophic factor; HGF, hepatocyte growth factor; FGF, fibroblast growth factor; LIF, leukemia inhibitory factor; IGF, insulin-like growth factor; IL, interleukin; EGF, epidermal growth factor; TGF, transforming growth factor; PDGF, platelet-derived growth factor: BMP, bone morphogenic protein; NCAM, neural cell adhesion molecule.

### EXAMPLES

### I. In Vitro (Cell-Based) Demonstrations of the Activity of Slit-2-N and Enhanced Efficacy of NGF/Slit-2-N Over NGF or Slit-2-N Alone

Cofractionation of Slit-2-N with the activity in calf brain extracts indicated that it was the active component in these extracts. We therefore purified recombinant hSlit-2-N (C-terminally histidine tagged hSlit-2 was expressed in COS cells, extracted with 1 M NaCl, and purified by WGA affinity chromatography, yielding pure Slit-2 and Slit-2-N; these proteins were then separated by nickel affinity chromatography on Ni+-NTA agarose (Invitrogen) following the manufacturer's instructions) and tested it in the E14 DRG assay (Wang et al., 1999, Cell 96, 771-784). Purified hSlit-2-N significantly stimulated axon elongation and branch formation, with a specific activity of ˜150 ng per unit, whereas a control fraction purified in parallel from COS cells transfected with the vector plasmid alone had no effect. Furthermore, the synergizing activity in the WGA flow-through fraction from calf brain extracts also potentiated the activity of hSlit-2-N. Full length hSlit-2 had no activity in the assay. Similar experiments demonstrate corresponding Slit-N amino terminal fragments of Slit-2 from other species including mouse and drosophia, as well as of Slit-1 and Slit-3 from human, mouse and drosophila also possess this activity.

### II. In Vivo Demonstrations of the Enhanced Efficacy of NGF/Slit-2-N Therapy Over NGF or Slit-2N Alone: Animal Model of Diabetic Polyneuopathy

Studies of the combination therapy in animal models demonstrate an enhanced restorative effect of the combination NGF/Slit-2-N therapy over either NGF or Slit-2-N alone in treating diabetic polyneuopathy. Study methodologies are described in Elias et al., 1998, Diabetes 47, 1637-1642.

Male mice (Ins. D^{d}1) have the murine MHO class I antigen D^{d} regulated by the human insulin gene in pancreatic β-cells (Jakobsen et al., 1981, Diabetes 30, 797-803). 6-month-old diabetic and nondiabetic litter mates are injected subcutaneously 3 times per week for 1 month with 1 mg/kg human recombinant NGF (GN0330 GI02AR), 1 mg/kg human recombinant Slit-2-N, a combination of 1 mg/kg human recombinant NGF and 1 mg/kg human recombinant Slit-2-N, or vehicle (10 mmol/l NaAcetate, pH 5.0/142 mmol/l NaCl) to determine the effect of combination NGF/Slit-2-N therapy on this neuropathy. Mice are examined by determining motor and sensory Cvs. Additional recordings are made directly from the sural nerve to assess C-fiber function.

Electrophysiology

H-Reflex. H-reflex and muscle waves (M-waves) are recorded from the plantar flexor muscles as substantially as described by Stanley, 1981, Exp Neurol 71, 497-505). At 2, 4, and 7 months of age, control and diabetic mice are anesthetized with ketamine (80 mg/kg)/xylazine (12 mg/kg). Their body temperature is maintained at 36-37° C. Monopolar needle electrodes (Nicolet, Madison, Wis. are used to record compound muscle potentials from the foot. One electrode is inserted into the plantar muscles of the foot, and the other is placed into the foot pad to serve as the reference electrode. A third needle electrode is inserted into the opposite hip to serve as a ground. The recording electrodes are connected to a differential AC amplifier and then to the interface of a MacLab Data Acquisition System (ADInstruments, Milfore, Mass.). The nerves to the plantar muscles are stimulated at two sites using percutaneous needle electrodes. Electrodes are placed in the medial aspect of the ankle to stimulate the tibial nerve and at the level of the sciatic notch to stimulate the sciatic nerve.

Square pulses of fixed duration (0.1 ms) are used to stimulate the nerves. Stimulus amplitude is slowly increased until either the H-reflex or the M-wave is first discernible. In general, an H-reflex can be obtained before the M-wave in nondiabetic control animals, but diabetic animals always produce an M-wave before the H-reflex. Stimulation is then increased until the maximum H-reflex is obtained. Each stimulation site is marked with indelible ink at the end of the study, and the distance between the sites is determined. The latency between the stimulus artifact and the first negative potential of the M-wave and the H-reflex is measured for recordings produced by stimulating at the hip and at the ankle. Motor nerve CV is calculated as follows: Motor nerve CV=(Distance between stimulation sites)/(Latency of M-wave (hip)−Latency of M-wave (ankle)). Sensory nerve conduction velocity is calculated as follows: Sensory nerve CV=(Distance between stimulation sites)/(Latency of H-reflex (hip)−Latency of H-reflex (ankle)).

Sural nerve recording. The C-fiber compound nerve potentials are obtained from the sural nerve using bipolar platinum hook electrodes. Mice are anesthetized with ketamine/xylazine, and body temperature is maintained as above. The sural nerve is exposed from the ankle to the knee, and the sciatic nerve is isolated between the knee and the hip. All divisions of the sciatic, except for the sural, are exposed and sectioned. The sciatic is isolated at the hip, sectioned, and the distal segment placed on bipolar stimulating electrodes. The sural nerve is crushed between the electrodes to provide a monopolar compound potential. A ground electrode is placed in the opposite hip. The entire exposed nerve is covered with a layer of Vaseline thinned with mineral oil to maintain temperature (35+0.5° C.) and prevent drying. The recording electrodes are connected via an AC differential amplifier to a MacLab Data Acquistion System. C-fiber compound potentials are evoked using 0.5 ms square pulses at amplitudes between 30 and 50 B. Between 16 and 32 C-fiber potential are recorded and averaged using an interstumulus interval of 15 s. The peak amplitude and integral of the compound C-fibers are calculated. CV of the most rapidly conducting C-fibers is determined by dividing the distance between the stimulating and recording electrodes by the latency.

Insulin (Dako, Santa Barbara, Calif., A564 lot 032) is detected using a Vectastain Elite ABC Kit, and DAB (diaminobenzidine tetrahydrochloride, Dako, S3000) is used to localize the peroxidase in the tissue sections.

Results. Blood glucose is elevated in the diabetic mice irrespective of treatment. Body weights are not significantly different after treatment. The compound motor and sensory CV's elicited from the sciatic nerve are decreased in diabetic mice and unchanged in animals treated with either NGF, Slit-2-N or the combination therapy. When recorded directly from the sural nerve, there is a reduction in C-fiber amplitude and integral in diabetic mice, indicating fewer fibers and asynchronous firing, whereas the CV is similar to that of the control. The decreased C-fiber amplitude and integral in control diabetic mice was normalized after a month of NGF or combination therapy. Results demonstrate enhanced restoration of C-fiber function with each NGF and Slit-2-N and a synergistic enhancement with the combination therapy.

### III. In Vivo Demonstrations of the Enhanced Efficacy of NGF/Slit-2-N Therapy Over NGF or Slit2-N Alone: Diabetic Polyneuopathy

Studies of the combination therapy in humans also demonstrate an enhanced restorative effect of the combination therapy over NGF alone in treating diabetic polyneuopathy. Study methodologies are described in Apfel SC; Kessler J A; Adomato B T; Litchy W J; Sanders C; Rask C A., Recombinant human nerve growth factor in the treatment of diabetic polyneuropathy. NGF Study Group, Neurology, 1998 September, 1(3):695-702 and summarized below.

Patients with symptomatic polyneuropathy randomly receive either placebo, NGF, Slit-2-N or combination NGF/Slit-2-N therapy for six months. Study subjects have stage 2 neuropathy as defined by Dyck et al., 1985, Brain 108, 861-880 & Dyck et al., 1992, Neurology 42, 1164-1170) and at least one symptom of small fiber neuropathy, at least one abnormal nerve conduction attribute in tow or more nerves, and quantitative sensory testing abnormalities for cooling and/or pain thresholds and are excluded if they have had clinically significant systemic disearse other than diabetes, active neoplastic disease, unstable proliferative retinopathy, or nondiabetic risk factors for neuropathy.

Eligible stubjects are randomly assigned to one of the four treatment groups in a 1:1:1:1 fashion. One group receives placebo (vehicle buffer), the second group receives rhNGF (Genentech Inc., South San Francisco) at a dose of 0.1 μg/kg in 150 μL, the third group receives rhSlit-2-N at a dose of 0.1 μg/kg in 150 μL, and the fourth group receives rhNGF and rhSlit-2-N, each at a dose of 0.1 μg/kg in 150 μL. Subjects receive injections subcutaneously three times per week for 6 consecutive months. Subjects and examiners are blinded as to whether they are receiving placebo, rhNGF, rhSlit-2-N or the combination.

Overall neuropathic impariment as determined by neurologic examination is assessed using the Neuopathy Impariment Score (NIS) (Dyck et al., 1995, Neurology 45, 1115-1121). A subscore of the NIS, impairments in the lower limbs (NISLL), is calculated separately as it reflects the site most affected in diabetic neuropathy.

Three different symptom assessments are used: the Neuropathy Symptom Profile (NSP) (Dyck et al., 1986, Neurology 36, 1300-1308), the Neuropathy Symptoms and Change (NSC), and a global symptom assessment.

Sensory perception are quantified using the CASE IV System (WR Medical Electronics Co., Stillwater, Minn.). Three different sensory modalities are measured, including cooling detection threshold (CDT), an intermediate response of pain from graded heating pulses (HP:5.0) and vibratory detection threshold (VDT) using techniques and algorithms as described in Dyck et al., 1987, Diabetes Care 10, 432-440; Dyck et al., 1993, Neurology 43, 1500-1508; Gruener et al., 1994, J Clin Neurophysiol 11, 568-588; Dyck et al, 1993, Neurology 43, 1508-1512; Dyck et al., 1996, J Neurol Sci 136, 54-63. A 4, 2 and 1 stepping algorithm is used for the CDT and VDT testing (Dyck et al, 1993, Neurology 43, 1508-1512). Thresholds are expressed as units of displacement (VDT) and change in ° C. (CDT). Heat perceived as pain is also expressed in ° C.

Nerve conduction studies are performed by American Board of Electromyography certified physicians according to a standardized uniform protocol. Recordings are conducted on the sural sensory and peroneal motor nerves in the legs as well as the ulnar motor and sensory and median sensory nerves in the arms for each subject. Unobtainable potentials are assigned a value of 0 for amplitude measurements and a value equal to the 99 percentile for latency measurements.

The following measures are considered as independent endpoints for efficacy: the quantitative neurologic examination (NISLL), quantitative measures of sensory function (CDT, VDT, and HP:5.0), and the symptom questionnaires (NSP, NSC and the global symptom assessment).

Efficacy of rhNGF, rhSlit-2-N and combination treatments are assessed by comparing endpoints pre- and posttreatment. The global symptom assessment reveals a strong beneficial effect of each of the rhNGF and the Slit-2-N on the subjects overall perception of their neuropathic symptomsand even stronger beneficial effect of the combination treatment (Table 4).

TABLE 4
Global Symptom Assessment

| Treatment | worsened | unchanged | improved |
|---|---|---|---|
| placebo | 45% | 45% | 10% |
| rhNGF | 1% | 26% | 73% |
| rhSlit-2-N | 1% | 25% | 74% |
| combination | 1% | 17% | 82% |

The CDT and HP:5.0 demonstrates improvement in both the rhNGF and rhSlit-2-N groups and even greater improvement in the combination treatment group, whereas the placebo group demonstrates no improvement (Table 5). The VDT reveals no significant differences between the groups.

TABLE 5

| Treatment | CDT (normal deviates) | HP5.0 (normal deviates) |
|---|---|---|
| placebo | −0.01 | −0.02 |
| rbNGF | −0.16 | −0.45 |
| rhSlit-2-N | −0.18 | −0.46 |
| combination | −0.24 | −0.60 |

Subjects receiving rhNGF or rhSlit-2-N demonstrate improvement in their quantitative neruologic examination (NISLL) compared with the placebo group, and those receiving the combination therapy improve even more markedly (Table 6). No significant differences are observed in the prevalence of symptoms in NSP, though there is improvement in the severity of symptoms as determined by the NSC, in both rhNGF and rhp40 treated groups and even more so in the combination treated group. Prospectively designated multiple endpoint analyses indicate that each of rhNGF, rhp40 and the combination are efficacious.

TABLE 6

| Treatment | CDT (normal deviates) | HP5.0 (normal deviates) |
|---|---|---|
| placebo | −0.01 | −0.02 |
| rhNGF | −0.16 | −0.45 |
| rhSlit-2-N | −0.18 | −0.46 |
| combination | −0.24 | −0.60 |

All publications and patent applications cited in this specification and all references cited therein are herein incorporated by reference as if each individual publication or patent application or reference were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. An isolated Slit-N polypeptide.

2. A Slit-N polypeptide according to claim 1, wherein the Slit-N polypeptide is selected from an hSlit-1-N, an hSlit-2-N and an hSlit-3-N polypeptide

3. A Slit-N polypeptide according to claim 1, contained in a pharmaceutical composition.

4. A Slit-N polypeptide according to claim 1, made by expressing a Slit protein in a cell, whereby the Slit protein is proteolytically processed to form the Slit-N polypeptide.

5. A recombinant polynucleotide comprising a coding region encoding a Slit-N polypeptide according to claim 1, said coding region flanked by fewer than 500 nucleotides of native flanking sequence.

6. A pharmaceutical composition comprising a therapeutically effective amount of a Slit-N polypeptide according to claim 1, and a pharmaceutically acceptable excipient.

7. A pharmaceutical composition comprising a therapeutically effective amount of a Slit-N polypeptide according to claim 1, and a pharmaceutically acceptable excipient, wherein the Slit-N polypeptide is selected from an hSlit-1-N, an hSlit-2-N and an hSlit-3-N polypeptide

8. A pharmaceutical composition comprising a therapeutically effective amount of a Slit-N polypeptide according to claim 1, and a pharmaceutically acceptable excipient, further comprising a therapeutically effective amount of a neuroactive agent other than the Slit-N polypeptide.

9. A pharmaceutical composition comprising a therapeutically effective amount of a Slit-N polypeptide according to claim 1, and a pharmaceutically acceptable excipient, further comprising a therapeutically effective amount of a neuroactive agent other than the Slit-N polypeptide, wherein the agent is NGF.

10. A method of promoting axon branching or sprouting, comprising contacting a neuron with a composition comprising an effective amount of a Slit-N polypeptide according to claim 1.

11. A method of treating a neuropathy comprising administering a composition comprising a therapeutically effective amount of a Slit-N polypeptide according to claim 1.
